(19) United States
(12) Patent Application Publication
(10) Pub. No.: US 2013/0046286 A1
(43) Pub. Date: Feb. 21, 2013

(54) NARROW HYSTERESIS NI-TI CORE WIRE FOR ENHANCED GUIDE WIRE STEERING RESPONSE

(71) Applicant: John A. Simpson, Carlsbad, CA (US)

(72) Inventor: John A. Simpson, Carlsbad, CA (US)

(73) Assignee: ABBOTT CARDIOVASCULAR SYSTEMS, Santa Clara, CA (US)

(21) Appl. No.: 13/211,796

(22) Filed: Aug. 17, 2011

(51) Int. Cl. A61M 25/09; C22F 1/00

(52) U.S. Cl. 604/528; 148/516; 148/527; 148/537

(57) ABSTRACT

Guide wire devices and methods for their manufacture. The guide wire devices described herein include an elongate guide wire member that includes at least one section fabricated from a nickel-titanium (Ni—Ti) alloy that exhibits an elevated plateau stress and a narrowed stress hysteresis profile (e.g., a plateau stress of about 500 MPa to about 820 MPa and a stress hysteresis width in a range from about 250 MPa to about 80 MPa). Raising the plateau stress and narrowing the stress hysteresis width of Ni—Ti used in a guide wire device can significantly improve the steerability of the guide wire device while maintaining the flexibility, durability, and kink resistance that is typical of superelastic Ni—Ti alloys.

### BACKGROUND

Guide wires are used to guide a catheter for treatment of intravascular sites such as PTCA (Percutaneous Transluminal Coronary Angioplasty), or in examination such as cardio-angiography. For example, a guide wire used in the PTCA is inserted into the vicinity of a target angiostenosis portion together with a balloon catheter, and is operated to guide the distal end portion of the balloon catheter to the target angiostenosis portion.

A guide wire needs appropriate flexibility, pushability and torque transmission performance for transmitting an operational force from the proximal end portion to the distal end, and kink resistance (resistance against sharp bending). To meet such requirements, superelastic materials such as a Ni—Ti alloy and high strength materials have been used for forming a core member (wire body) of a guide wire.

Near equi-atomic nickel-titanium alloys are known to exhibit “pseudo-elastic” behavior when given certain cold working processes or cold working and heat treatment processes following hot working Pseudo-elasticity can be further divided into two subcategories: “non-linear” pseudo-elasticity and “linear” pseudo-elasticity. “Non-linear” pseudo-elasticity is sometimes used by those in the industry synonymously with “superelasticity.”

“Non-linear” pseudo-elastic Ni—Ti alloy exhibits upwards of 8% elastic strain (fully-recoverable deformation) by virtue of a reversible, isothermal stress-induced martensitic transformation. Non-linear pseudo-elasticity is known to occur due to a reversible phase transformation from austenite to martensite, the latter more precisely called “stress-induced martensite” (SIM). At room or body temperature and under minimal stress the material assumes a crystalline microstructure structure known as austenite. As the material is stressed, it remains in the austenitc state until it reaches a threshold of applied stress (a.k.a. the “plateau stress” or the “upper plateau stress”), beyond which the material begins to transform into a different crystal structure known as martensite. Upon removal of the applied stress, the martensite typically transforms back to the original austenite structure with an accompanying return to essentially zero strain (i.e., the original shape is restored). Linear pseudo-elastic Ni—Ti exhibits no such upper plateau stress.

“Linear” pseudo-elastic Ni—Ti typically results from cold working the material (e.g., by permanently deforming the material such as by wire-drawing) without subsequent temperature treatment (i.e., annealing). Residual permanent deformation, i.e., “cold work,” tends to stabilize the martensitic structure so its reversion back to austenite is retarded or altogether blocked. With increasing levels of permanent deformation, the otherwise austenitic material becomes fully martensitic at room and body temperature, and further permanent deformation serves to progressively raise its yield strength. The almost complete disappearance of austenite via cold work altogether eliminates the plateau (austenite to martensite transformation) on the stress strain curve, resulting in a conventional metallic stress strain curve featuring a classic linear slope until its yield strength is reached.

### BRIEF SUMMARY

The present disclosure describes guide wire devices and methods for their manufacture. The guide wire devices described in the present disclosure include an elongate guide wire member that includes at least one section fabricated from a nickel-titanium (Ni—Ti) alloy that exhibits an elevated upper plateau stress and a stress hysteresis width (i.e., the difference between the upper plateau stress and a lower plateau stress) that is narrowed relative to conventionally processed Ni—Ti. In one example, the alloy exhibits a plateau stress of about 500 MPa to about 820 MPa and a stress hysteresis width in a range from about 250 MPa to about 80 MPa. Raising the plateau stress and narrowing the stress hysteresis width of Ni—Ti used in a guide wire device can significantly improve the steerability of the guide wire device while maintaining the flexibility, durability, and kink resistance that is typical of superelastic Ni—Ti alloys.

According to one embodiment of the present disclosure, the elevated upper plateau stress and the narrowed stress hysteresis width of the Ni—Ti alloy are each imparted by about 30 to 50% cold work and heat treatment at a temperature of at least about 550K to about 750K for about 1 minute to about 30 minutes.

In yet another embodiment, a method for fabricating a guide wire device that includes a Ni—Ti alloy having an elevated upper plateau stress and a narrowed stress hysteresis width is disclosed. The method includes (1) providing an elongate guide wire member that includes a proximal section and a distal section, wherein at least the distal section is fabricated from a Ni—Ti alloy, (2) cold working at least a portion of the distal section to yield a cold worked Ni—Ti alloy that exhibits at least about 30% cold work, and (3) heat treating at least the cold worked portion at a temperature of at least about 550K for about 1 minute to about 30 minutes. The extent of the cold working and the duration and temperature of the heat treatment are selected to yield a Ni—Ti alloy that exhibits an elevated upper plateau stress of at least about 500 MPa and a narrowed stress hysteresis width of about 250 MPa.

In a more detailed embodiment, the method further includes (4) disposing a helical coil section about at least a distal end portion of the distal section, (5) joining the helical coil to the elongate guide wire member at a proximal location, (6) forming a rounded cap section on a distal end of the helical coil, and (7) applying at least one lubricious outer coating layer over at least a portion of the elongate guide wire member to form the guide wire device.

These and other objects and features of the present disclosure will become more fully apparent from the following description and appended claims, or may be learned by the practice of the embodiments of the invention as set forth hereinafter.

### BRIEF DESCRIPTION OF THE DRAWINGS

To further clarify the above and other advantages and features of the present disclosure, a more particular description of the invention will be rendered by reference to specific embodiments thereof which are illustrated in the appended drawings. It is appreciated that these drawings depict only illustrated embodiments of the invention and are therefore not to be considered limiting of its scope. The present disclosure will be described and explained with additional specificity and detail through the use of the accompanying drawings in which:

FIG. 1 illustrates stress-strain curves for stainless steel, a linear pseudoelastic Ni—Ti alloy, and a superelastic (i.e., non-linear pseudoelastic) Ni—Ti alloy;

FIG. 2 depicts a series of stress-strain curves for a Ni—Ti alloy illustrating the effect of raising the upper plateau stress and narrowing of the stress hysteresis profile with increasing amounts of cold work followed by limited heat treatment;

FIG. 3 illustrates a load-strain curve for a Ni—Ti alloy processed according to an embodiment of the present invention;

FIG. 4 illustrates a partial cut-away view of a guide wire device according to one embodiment of the present invention; and

FIG. 5 is a side elevation view, in partial cross-section, of a delivery catheter within a body lumen having a stent disposed about the delivery catheter according to an embodiment of the present disclosure.

### DETAILED DESCRIPTION

### I. Introduction

The present disclosure describes guide wire devices and methods for their manufacture. The guide wire devices described in the present disclosure include an elongate guide wire member that includes at least one section fabricated from a nickel-titanium (Ni—Ti) alloy that exhibits an elevated upper plateau stress and a stress hysteresis width (i.e., the difference between the upper plateau stress and a lower plateau stress) that is narrowed relative to conventionally processed Ni—Ti. In one example, the alloy exhibits a plateau stress of about 500 MPa to about 820 MPa and a stress hysteresis width in a range from about 250 MPa to about 80 MPa. Raising the plateau stress and narrowing the stress hysteresis width of Ni—Ti used in a guide wire device can significantly improve the steerability of the guide wire device while maintaining the flexibility, durability, and kink resistance that is typical of superelastic Ni—Ti alloys.

Because guide wire devices are designed to track through a patient's vasculature, for example, guide wires may be quite long (e.g., about 150 cm to about 300 cm in length) and thin. Guide wire devices need to be long enough to travel from an access point outside a patient's body to a treatment site and narrow enough to pass freely through the patient's vasculature. For example, a typical guide wire device has an overall diameter of about 0.2 mm to about 0.5 mm for coronary use. Larger diameter guide wires, up to about 1.0 mm, may be employed in peripheral arteries and other body lumens. The diameter of the guide wire device affects its flexibility, support, and torque. Thinner wires are more flexible and are able to access narrower vessels while larger diameter wires offer greater support and torque transmission.

Typical guide wire devices are constructed of a superelastic binary Ni—Ti distal core section and a stainless steel proximal core section. The distal and proximal sections are typically joined together by either a mechanical or a welded joint. The distal core section is made from superelastic Ni—Ti because it has extreme resistance to permanent deformation (approx. 8% elastic strain limit, vs. about 1% for high strength stainless steel), so it is difficult to kink even when advanced through extremely tortuous vasculature or when prolapsed during use.

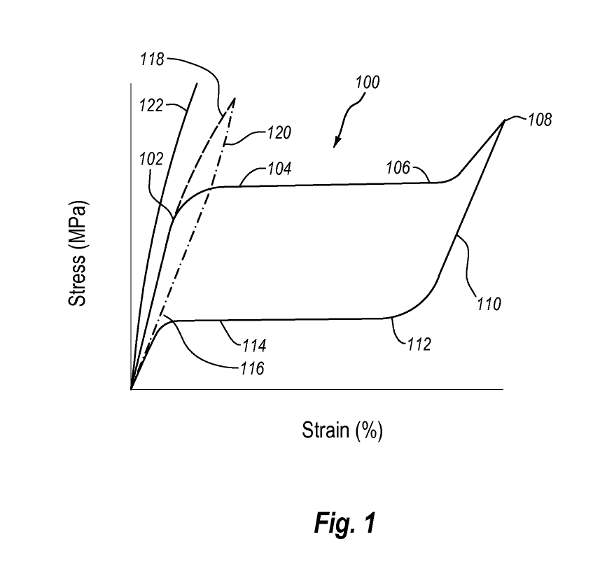

To illustrate the foregoing, FIG. 1 illustrates idealized stress-strain curves for superelastic Ni—Ti 100 (i.e., non-linear pseudoelastic Ni—Ti), linear pseudo elastic Ni—Ti 118/120, and, for comparison, stainless steel 122. The stress/strain relationship is plotted on x-y axes, with the x axis representing strain and the y axis representing stress.

In curve 100, when stress is applied to a specimen of Ni—Ti alloy exhibiting non-linear pseudoelastic characteristics at a temperature at or above where the material is in the austenite phase, the Ni—Ti alloy deforms elastically in region 102 until it reaches a particular stress level where the alloy then begins to undergo a stress-induced phase transformation from the austenitic phase to the martensitic phase (i.e., the stress-induced martensite phase). As the phase transformation progresses, the Ni—Ti alloy undergoes significant increases in strain with little or no corresponding increases in stress. On curve 100, this is represented by the upper, nearly flat stress plateau 104. The strain increases while the stress from continued deformation remains essentially constant until the transformation of the austenitic phase to the martensitic phase is complete at approximately region 106. Thereafter, further increase in stress is necessary to cause further deformation to point 108. The Ni—Ti alloy in the martensitic phase first yields elastically upon the application of additional stress and then plastically with permanent residual deformation (not shown).

If the load on the specimen is removed before any permanent deformation has occurred, the Ni—Ti alloy in the martensitic phase elastically recovers and transforms back to the austenitic phase. The reduction in stress first causes a decrease in strain along region 110. As stress reduction reaches the level at which the martensitic phase begins to transform back into the austenitic phase at region 112, the stress level in the specimen remains essentially constant along lower plateau 114 (but less than the constant stress level at which the austenitic crystalline structure transforms to the martensitic crystalline structure until the transformation back to the austenitic phase is complete); i.e., there is significant recovery in strain with only negligible corresponding stress reduction. This is represented in curve 100 by the lower stress plateau 114. As used herein, the difference between the upper plateau 104 and the lower plateau 114 is referred to as the “stress hysteresis width,” the “hysteresis width,” or variations thereof.

After the transformation back to austenite is complete, further stress reduction results in elastic strain reduction along region 116. This ability to incur significant strain at relatively constant stress upon the application of a load and to recover from the deformation upon the removal of the load is commonly referred to as non-linear pseudoelasticity (or superelasticity).

For comparison to the superelastic stress-strain curve 100, FIG. 1 also includes a curve 118/120 representing the idealized behavior of linear pseudoelastic Ni—Ti alloy. Curve 118/120 does not contain any flat plateau stresses found in curve 100. This stands to reason since the Ni—Ti alloy of curve 118/120 remains in the martensitic phase throughout and does not undergo any phase change. To that end, curve 118/120 shows that increasing stress yields a proportional increase in reversible strain, and a release of stress yields a proportional decrease in strain.

For reference, FIG. 1 also includes curve 122 which is the elastic behavior of a standard 316L stainless steel. Stress is incrementally applied to the steel and, just prior to the metal deforming plastically, decrementally released. In contrast to superelastic Ni—Ti, stainless steel can tolerate only relatively small amounts of strain without experiencing plastic deformation. That is, stainless steel and metals like it are likely to permanently deform if even slightly deformed, whereas both linear elastic and superelastic Ni—Ti can be deformed relatively significantly without permanently deforming.

Despite the advantages of a superelastic Ni—Ti distal guide wire core (e.g., the ability to bend quite dramatically without causing permanent deformation), typical guide wires from Ni—Ti generally do not transmit applied torque from the proximal shaft to the distal tip as well as all-stainless-steel “core to tip” guide wire designs. This is the case, in part, because the shear modulus or “modulus of rigidity” of binary Ni—Ti is substantially lower than that of stainless steel. As a result, superelastic Ni—Ti tends to wind up and store twist as opposed to transmitting torque smoothly from end-to-end.

For this reason, the grind profiles of superelastic Ni—Ti core wires are typically larger than those of stainless steel, and there is an inherent limit to the torque transmission that can be obtained with superelastic Ni—Ti core wires without exceeding product profile requirements. A purposefully large grind profile on a superelastic Ni—Ti core wire means that navigation through tortuous vasculature often induces levels of bending strain that cause the normally austenitic structure to locally transform into martensite. This occurs wherever the associated stress levels exceed the upper plateau stress (region 104 in FIG. 1). It is advantageous that such transformation is reversible upon unloading, because this enables the core wire to return to its originally straight condition when other materials like stainless steel would be permanently deformed (kinked). In most locations, permanent deformation of a core wire can significantly impair guide wire performance, particularly torsional response and thus steerability, because for the user to attain complete rotation of the distal tip the deformed section must be forced to reverse-bend before revolving back to its initial deformed orientation. Assuming the user is able to attain one complete rotation at all, a permanently deformed guide wire will likely “whip” or resist rotation initially and then rotate abruptly rather than rotate freely.

Referring still to FIG. 1, because the stress (vertical) axis relates to force, and the strain axis (horizontal) relates to distance, the area bounded by curve 100 (i.e., the stress hysteresis loop) is a measure of energy loss (force×distance=work or energy). That is, the area under the upper plateau stress 104 represents the strain energy imparted to the material via tensile loading, while the area under the lower plateau stress 114 represents the strain energy returned by the material during tensile unloading, and the area bounded by curve 100 represents energy that is dissipated to the environment in the form of heat.

The stress hysteresis inherent with conventional superelastic Ni—Ti can be a significant detriment to the steering performance of guide wires. Its negative impact is apparent when bending is severe enough to induce stresses within the core wire that exceeds the material's upper plateau stress 104 and, simultaneously, the user attempts to rotate the distal tip by applying torque to the guide wire's proximal shaft. In such a situation, the area bounded by the upper plateau 104 and the lower plateau 114 represents an energy loss that must be overcome via additional applied torque in order to steer the guide wire. That is, because some of the energy of torque is taken up in the form of heat, the user must apply more rotational energy in order to accomplish the desired steering movement.

In contrast, the use of a truly elastic core material without hysteresis, such as stainless steel, would exhibit substantially no energy loss and thus, in a similar scenario, would be expected to require less applied energy to rotate. However, as mentioned above, stainless steel is susceptible to permanent deformation if the bending strains exceed the elastic limit of the material.

### II. Narrow Hysteresis Ni—Ti

As an alternative to conventional superelastic Ni—Ti or stainless steel, the present disclosure relates to the use of a core Ni—Ti alloy material that is processed so as to have an elevated upper plateau stress, an elevated lower plateau stress, and a narrowed stress hysteresis width, as compared to typically processed superelastic Ni—Ti. Such a material can be expected to exhibit improved steering response because steering forces are less likely induce transformation of austenite to martensite (due to the elevated upper plateau stress) and, if the material is overstressed during steering causing the conversion of austenite to martensite, the material would be expected to require less applied torque to rotate because of the reduced energy loss by virtue of the narrowed hysteresis loop.

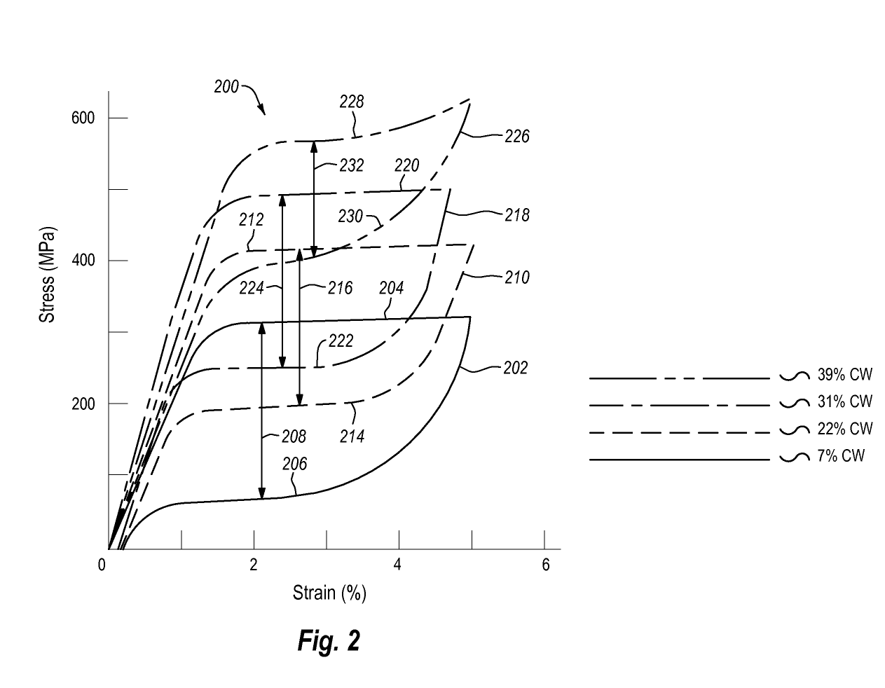

Referring now to FIG. 2, a series of stress-strain curves (202, 210, 218, and 226) for a Ni—Ti alloy are depicted illustrating some of the potential advantages of using a Ni—Ti alloy material that is processed so as to have an elevated upper plateau stress and a narrowed hysteresis width. In particular, FIG. 2 illustrates the effects on a Ni—Ti alloy of increasing amounts of cold work followed by limited heat treatment.

The Ni—Ti alloy samples corresponding to the curves in FIG. 2 represent the effect of ˜7% cold work (curve 202), ˜22% cold work (curve 210), −31% cold work (curve 218), and ˜39% cold work (curve 226) followed by limited heat treatment at ˜623K for approximately 30 minutes. As used herein, the term “limited heat treatment” refers to heat treatment where either one of or both of the temperature or the duration of heat treatment are insufficient to completely reverse the effects of cold work. That is, cold work followed by limited heat treatment results in a partially annealed microstructure that retains some linear pseudoelastic characteristics but that is predominantly superelastic. It is likely that the observed behavior of the partially annealed metal samples illustrated in FIG. 2 results from a microstructure that includes very fine grains or dislocation cell structures (subgrains). The numerous boundaries of the very fine grains or dislocation cell structures substantially influence the stress-induced transformation from an entirely austenitic structure into martensite and also substantially influence the corresponding reversion upon stress removal.

As depicted in FIG. 2, as the amount of cold work is increased from ˜7% (curve 202) to ˜39% (curve 226) (followed by limited heat treatment), the upper plateau stress increases and the hysteresis width narrows. Curve 202 has an upper plateau stress 204 of about 300 MPa, a lower plateau stress 206 of about 50 MPa, and a hysteresis width 208 of about 250 MPa. Curve 210 has an upper plateau stress 212 of about 400 MPa, a lower plateau stress 214 of about 175 MPa, and a hysteresis width 208 of about 225 MPa. Curve 218 has an upper plateau stress 220 of about 500 MPa, a lower plateau stress 222 of about 250 MPa, and a hysteresis width 224 of about 250 MPa. And Curve 226 has an upper plateau stress 228 of about 575 MPa, a lower plateau stress 230 of about 450 MPa, and a hysteresis width 208 of about 125 MPa. All measurements illustrated in FIG. 1 were performed at about 40° C. This is within the temperature range at which the subject guide wire device would be expected to be used (i.e., about 35° C. to about 40° C.).

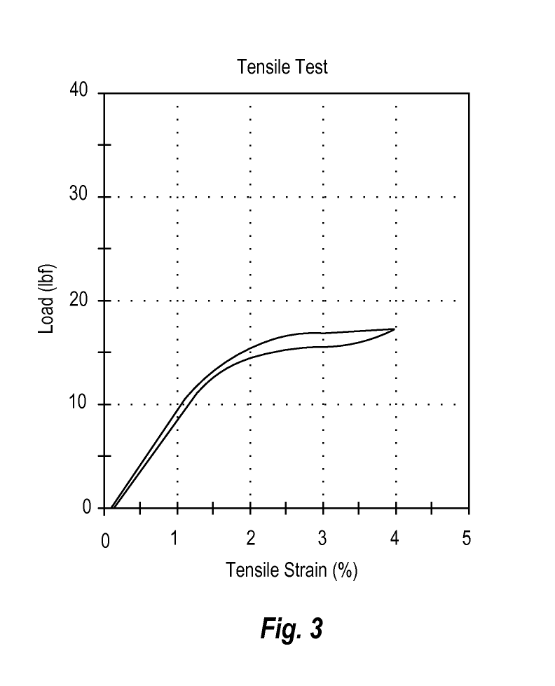

FIG. 3 illustrates a load-strain curve for a Ni—Ti alloy sample processed according to an embodiment of the present invention. The subject sample corresponding to the curve in FIG. 3 is a binary Ni—Ti alloy wire with a diameter of about 0.35 mm (i.e., 0.01344 inch). The Ni—Ti alloy sample of FIG. 3 has an upper plateau load of 16.88 lbf and a lower plateau load of 15.23 lbf when tested at room temperature. The loads (lbf) can be converted to stress values (psi) by dividing load by [pi)×(dia)̂2]/4 where dia=0.01344. The Ni—Ti alloy sample of FIG. 3 has an upper plateau stress of 118,985 psi and a lower plateau stress of 107,355 psi. Psi can be converted to MPa by dividing psi by 145.0377. As such, the Ni—Ti alloy processed by cold working followed by limited heat treatment has an elevated upper plateau stress of about 820 MPa and a narrowed stress hysteresis width of about 80 MPa.

In one embodiment, the Ni—Ti alloy employed in the subject guide wire device has an elevated upper plateau stress and a narrowed stress hysteresis width imparted by about 30 to 50% cold work and heat treatment at a temperature of at least about 550K to about 750K for about 1 minute to about 30 minutes.

In another embodiment, the Ni—Ti alloy employed in the subject guide wire device has an elevated upper plateau stress and a narrowed stress hysteresis width imparted by at least about 30% cold work and heat treatment at a temperature of at least about 550K for about 1 minute to about 30 minutes.

In yet another embodiment, the Ni—Ti alloy employed in the subject guide wire device has an elevated upper plateau stress and a narrowed stress hysteresis width imparted by about 40% cold work and heat treatment at a temperature of about 670K to about 725K for about 30 minutes.

Such cold work amounts and heat treatment temperatures and duration yield an elevated upper plateau stress in a range from about 500 MPa to about 820 MPa, or an elevated upper plateau stress of about 550 MPa and a narrowed stress hysteresis width of about 150 MPa, or an elevated upper plateau stress of about 820 MPa and a narrowed stress hysteresis width of about 80 MPa.

In some embodiments, the Ni—Ti alloys used in the guide wire devices described herein exhibit an elevated upper plateau stress of at least about 500 MPa and a narrowed stress hysteresis width of about 250 MPa. As compared to conventionally processed Ni—Ti (e.g., curve 100 in FIG. 1 or curve 202 in FIG. 2), a Ni—Ti alloy having an elevated upper plateau stress of at least about 500 MPa and a narrowed stress hysteresis width of about 250 MPa or less exhibits improved steering response because steering forces are less likely induce transformation of austenite to martensite (due to the elevated upper plateau stress) and, if the material is overstressed during steering causing the conversion of austenite to martensite, the material would be expected to require less applied torque to rotate because of the reduced energy loss by virtue of narrowing the hysteresis loop.

In one embodiment, the Ni—Ti alloy used in the subject guide wire device includes about 54.5 wt % to about 57 wt % Ni and a balance of Ti. In another embodiment, the Ni—Ti alloy used in the subject guide wire device includes about 30 to about 52% titanium and the balance nickel and up to 10% of one or more other alloying elements. The other alloying elements may be selected from the group consisting of iron, cobalt, vanadium, platinum, palladium, copper, niobium, tantalum and combinations of the foregoing. The alloy can contain up to about 10% copper and vanadium and up to 3% of iron and cobalt and up to about 25 or 30% of the other alloying elements. In yet another embodiment, the Ni—Ti alloy used in the subject guide wire device includes about 50.2 at % Ni and about 49.8 at % Ti.

### III. Guide Wire Devices

As discussed in greater detail elsewhere herein, guide wire devices are typically made from stainless steel, a conventionally processed super elastic Ni—Ti alloy, or a combination of the two. For a given wire diameter, stainless steel is quite a bit stiffer than superelastic Ni—Ti and is generally better at transmitting torque. Nevertheless, stainless steel is susceptible to kinking while passing through tortuous anatomy. In contrast, superelastic Ni—Ti is much less susceptible to kinking but it is not as effective at transmitting applied torque.

In ordinary applications, differences in flexibility between two materials can be readily compensated for by dimensional alterations. That is, for example, the tendency to “wind up” that is typical of conventionally processed superelastic Ni—Ti can ordinarily be compensated for by increasing the diameter of the wire in order to attain equivalent deflection behavior when compared to a stiffer wire material. However, guide wire devices typically face inherent dimensional constraints that are imposed by the overall product profile, by the allowable space within overlying coils or polymeric jacketing, and/or the size of the anatomy to be accessed. For this reason, the Ni—Ti alloys having an elevated upper plateau stress and a narrowed stress hysteresis discussed herein significantly expand the maximum range of torsional or bending stiffness that can be achieved in a Ni—Ti guide wire of a given profile.

In one embodiment, a guide wire device includes an elongate guide wire member having a proximal section and a distal section. At least a portion of the elongate guide wire member is fabricated from a cold worked and partially heat treated Ni—Ti alloy that, as a result of the cold work and partial heat treatment (e.g.,. at least about 30% cold work and partial heat treatment at a temperature of at least about 550K), displays an elevated upper plateau stress of at least about 500 MPa and a narrowed stress hysteresis width of about 250 MPa, as measured at a temperature of about 40° C.

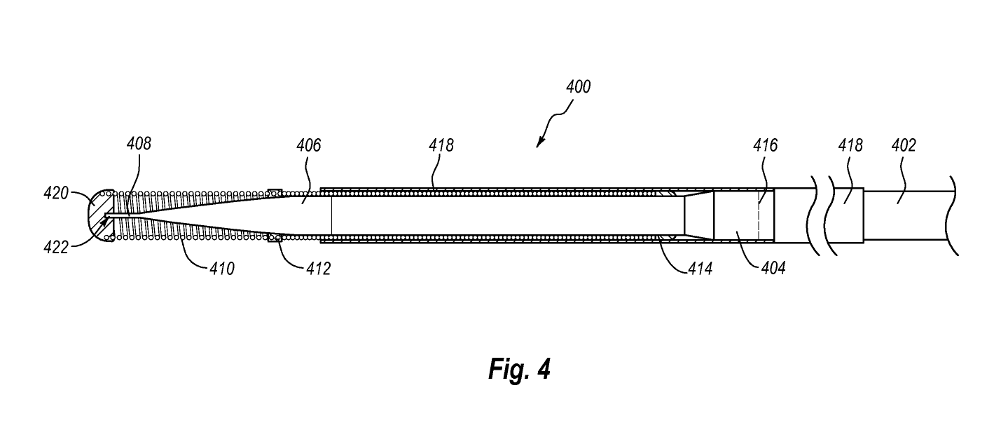

Referring now to FIG. 4, a partial cut-away view of an example of a guide wire device 400 is illustrated. The guide wire device 400 may be adapted to be inserted into a patient's body lumen, such as an artery or another blood vessel. The guide wire device 400 includes an elongate guide wire member that is made up of an elongated proximal portion 402 and a distal portion 404. In one embodiment, both the elongated proximal portion 402 and the distal portion 404 may be formed from a Ni—Ti alloy. In another embodiment, the elongated proximal portion 402 may be formed from a first material such as stainless steel (e.g., 316L stainless steel) or a Ni—Ti alloy and the distal portion may be formed from a second material such as a Ni—Ti alloy. In embodiments where the elongated proximal portion 402 and the distal portion 404 are formed from different materials, the elongated proximal portion 402 and the distal portion 404 may be joined to one another via a welded joint 416 that joins the proximal portion 402 and the distal portion 404 into a torque transmitting relationship.

In one embodiment, selected portions of the guide wire device 400 or the entire guide wire device 400 may be processed by cold working one or more Ni—Ti alloy portions followed by limited heat treatment to yield one or more Ni—Ti alloy portions that exhibit an elevated upper plateau stress of at least about 500 MPa and a narrowed stress hysteresis width of about 250 MPa or less. As discussed elsewhere herein, increasing levels of cold-work followed by limited heat treatment raises the upper plateau stress and narrows the stress hysteresis width, resulting in a Ni—Ti alloy material that torques more like stainless steel (or a similar material) yet retains the durability and kink resistance of super elastic Ni—Ti.

In various embodiments, the distal portion, the elongated proximal portion, or both may be cold worked to exhibit about 30% to about 50% cold work, about 30%, or about 40% cold work followed by heat treatment at a temperature in a range of 550K to about 750K for about 1 minute to about 30 minutes, 670K to about 725K for about 30 minutes, or about 550K for about 1 minute to about 30 minutes. Depending on the amount of cold work and the temperature and duration of the heat treatment, such a combination of cold work and heat treatment may yield a Ni—Ti alloy that exhibits an elevated upper plateau stress in a range from about 500 MPa to about 820 MPa and a narrowed stress hysteresis width in a range of about 250 MPa to about 80 MPa

Referring again to FIG. 4, the distal portion 404 has at least one tapered section 406 that, in the illustrated embodiment, becomes smaller in the distal direction. The length and diameter of the tapered distal core section 406 can, for example, affect the trackability of the guide wire device 400. Typically, gradual or long tapers produce a guide wire device with less support but greater trackability, while abrupt or short tapers produce a guide wire device that provides greater support but also greater tendency to prolapse (i.e., kink) when steering. The length of the distal end section 406 can, for example, affect the steerability of the guide-wire device 400. In one embodiment, the distal end section 406 is about 10 cm to about 40 cm in length.

In the illustrated embodiment, the tapered distal core section 406 may further include a shapeable end section 408. Such shapeable end sections can be integral to the guide wire device 400 as shown, or they can be a separate piece (not shown) that is included as part of the distal end of the guide wire device 400. Having a shapeable distal end section 408 can allow a practitioner to shape the distal and of the guide wire device 400 to a desired shape (e.g., a J-bend) for tracking through the patient's vasculature.

As illustrated in FIG. 4, the guide wire device 400 includes a helical coil section 410. The helical coil section 410 affects support, trackability, and visibility of the guide wire device and provides tactile feedback. Preferably, the most distal section of the helical coil section 410 is made of radiopaque metal such as platinum or palladium alloys to facilitate the observation thereof while it is disposed within a patient's body. As illustrated, the helical coil section 410 is disposed about at least a portion of the distal portion 404 and has a rounded, atraumatic cap section 420 on the distal end thereof. The helical coil section 410 is secured to the distal portion 404 at proximal location 414 and at intermediate location 412 by a suitable technique such as, but not limited to, soldering, brazing, welding, or adhesive.

In one embodiment, portions of the guide wire device 400 are coated with a coating 418 of lubricous material such as polytetrafluoroethylene (PTFE) (sold under the trademark Teflon by du Pont, de Nemours & Co.) or other suitable lubricous coatings such as the polysiloxane coatings, polyvinylpyrrolidone (PVP), and the like.

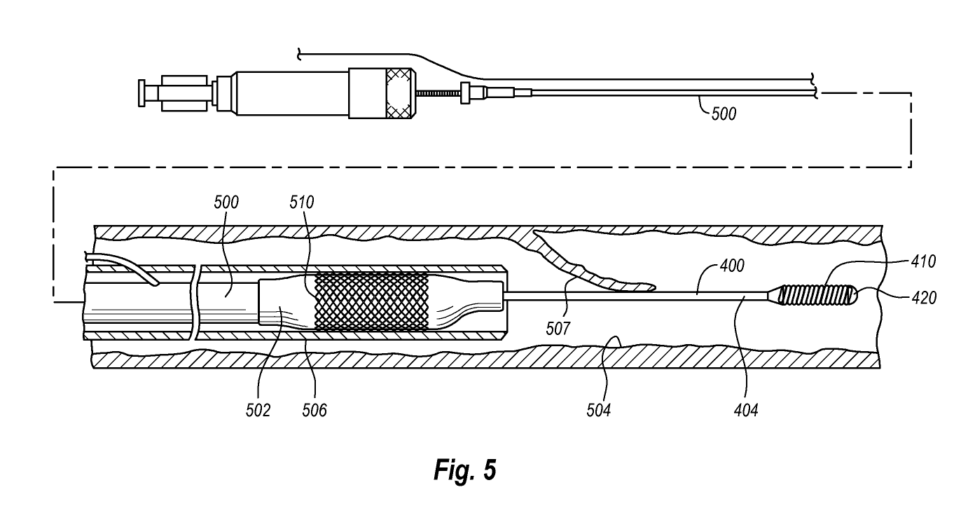

Referring now to FIG. 5, a guide wire device 400 is shown configured to facilitate deploying a stent 510. FIG. 5 provides more detail about the manner in which a guide wire device 400 may be used to track through a patient's vasculature where it can be used to facilitate deployment of a treatment device such as, but not limited to the stent 510. FIG. 5 illustrates a side elevation view, in partial cross-section, a delivery catheter 500 having a stent 510 disposed thereabout according to an embodiment of the present disclosure. The portion of the illustrated guide wire device 400 that can be seen in FIG. 5 includes the distal portion 404, the helical coil section 410, and the atraumatic cap section 420. The delivery catheter 500 has an expandable member or balloon 502 for expanding the stent 510, on which the stent 510 is mounted, within a body lumen 504 such as an artery.

The delivery catheter 500 may be a conventional balloon dilatation catheter commonly used for angioplasty procedures. The balloon 502 may be formed of, for example, polyethylene, polyethylene terephthalate, polyvinylchloride, nylon, or another suitable polymeric material. To facilitate the stent 510 remaining in place on the balloon 502 during delivery to the site of the damage within the body lumen 504, the stent 510 may be compressed onto the balloon 502. Other techniques for securing the stent 510 onto the balloon 502 may also be used, such as providing collars or ridges on edges of a working portion (i.e., a cylindrical portion) of the balloon 502.

In use, the stent 510 may be mounted onto the inflatable balloon 502 on the distal extremity of the delivery catheter 500. The balloon 502 may be slightly inflated to secure the stent 510 onto an exterior of the balloon 502. The catheter/stent assembly may be introduced within a living subject using a conventional Seldinger technique through a guiding catheter 506. A guide wire 508 may be disposed across the intended arterial section 507 and then the catheter/stent assembly may be advanced over the guide wire 508 within the body lumen 504 until the stent 510 is directly under the detached lining 507. For example, the guide wire 508 may be made from a superelastic nickel-titanium alloy, or another suitable material. The balloon 502 of the catheter 500 may be expanded, expanding the stent 510 against the interior surface defining the body lumen 504 by, for example, permanent plastic deformation of the stent 210. When deployed, the stent 510 holds open the body lumen 504 after the catheter 500 and the balloon 502 are withdrawn.

### IV. Methods for Fabricating a Guide Wire Device

In another embodiment, a method for fabricating a guide wire device is disclosed. The method includes (1) fabricating an elongate guide wire member having a proximal section and a distal section, wherein at one of the proximal section or the distal section is fabricated from a nickel-titanium (Ni—Ti) alloy, (2) cold working at least a portion of the Ni—Ti alloy to yield a cold worked section that exhibits at least about 30% cold work, and (3) heat treating the cold worked portion at a temperature of at least about 550K for about 1 minute to about 30 minutes. The amount of cold working followed by the limited heat treatment are selected to yield a Ni—Ti alloy that exhibits an elevated upper plateau stress of at least about 500 MPa and a narrowed stress hysteresis width of about 250 MPa or less.

In yet another embodiment, a method for fabricating a guide wire device that includes a Ni—Ti alloy having an elevated upper plateau stress and a narrowed stress hysteresis width is disclosed. The method includes (1) providing an elongate guide wire member that includes a proximal section and a distal section, wherein at least the distal section is fabricated from a Ni—Ti alloy, (2) cold working at least a portion of the distal section to yield a cold worked Ni—Ti alloy that exhibits at least about 30% cold work, and (3) heat treating at least the cold worked portion at a temperature of at least about 550K for about 1 minute to about 30 minutes. The extent of the cold working and the duration and temperature of the heat treatment are selected to yield a Ni—Ti alloy that exhibits an elevated upper plateau stress of at least about 500 MPa and a narrowed stress hysteresis width of about 250 MPa.

The method further includes (4) disposing a helical coil section about at least a distal end portion of the distal section, (5) joining the helical coil to the elongate guide wire member at a proximal location, (6) forming a rounded cap section on a distal end of the helical coil, and (7) applying at least one lubricious outer coating layer over at least a portion of the elongate guide wire member to form the guide wire device.

In one embodiment, the elongate guide wire member can be fabricated from a billet or ingot of the Ni—Ti alloy using at least one of drawing, swaging or grinding. Suitable examples of cold working procedures that can be used to cold work either selected sections of the elongate guide wire member or the whole elongate guide wire member include, but are not limited to, drawing, high force flattening, stamping, rolling, calendaring, and combinations thereof

In one embodiment, the methods disclosed herein further include (a) fabricating at least the distal section of the elongate guide wire member from a Ni—Ti alloy, (b) cold working the distal section of the elongate guide wire member to yield a Ni—Ti alloy distal section that exhibits at least about 30% cold work, and (c) heat treating the distal section at a temperature of at least about 550K for about 10 minutes to about 30 minutes to yield a Ni—Ti alloy distal section having an elevated upper plateau stress of at least about 500 MPa and a narrowed stress hysteresis width of about 250 MPa.

In another embodiment, the methods disclosed herein include (a) fabricating the proximal and distal sections of the elongate guide wire member from a Ni—Ti alloy, (b) cold working at least a portion the elongate guide wire member to yield a cold worked Ni—Ti alloy that exhibits at least about 30% cold work, and (c) heat treating the elongate guide wire member at a temperature of at least about 550K for about 10 minutes to about 30 minutes to yield a Ni—Ti alloy that exhibits an elevated upper plateau stress of at least about 500 MPa and a narrowed stress hysteresis width of about 250 MPa.

In one embodiment, the guide wire devices disclosed herein include at least one Ni—Ti alloy section that exhibits an elevated upper plateau stress in a range from about 500 MPa to about 820 MPa, or an elevated upper plateau stress in a range of about 500 MPa to about 820 MPa and a narrowed stress hysteresis width in a range of about 250 MPa to about 80 MPa.

In another embodiment, the methods for making guide wire devices disclosed herein cold working the cold worked section to yield a cold worked Ni—Ti alloy that includes about 30% to about 50% cold work, and heat treating at least the cold worked section at a temperature of about 550K to about 750K for about 10 minutes to about 30 minutes, or cold working the cold worked section to yield a cold worked Ni—Ti alloy that includes about 40% cold work, and heat treating at least the cold worked portion at a temperature of about 670K to about 725K for about 30 minutes.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A guide wire device, comprising:
an elongate guide wire member having a proximal section and a distal section, wherein at least a portion of the elongate guide wire member is fabricated from a nickel-titanium (Ni—Ti) alloy that exhibits an elevated upper plateau stress of at least about 500 MPa and a stress hysteresis width between the upper plateau stress and a lower plateau stress of about 250 MPa or less.

2. The guide wire device of claim 1, wherein the elevated upper plateau stress and the narrowed stress hysteresis width of the Ni—Ti alloy are each imparted by about 30 to 50% cold work and heat treatment at a temperature of at least about 550K to about 750K for about 1 minute to about 30 minutes.

3. The guide wire device of claim 2, wherein the elevated upper plateau stress and the narrowed stress hysteresis width of the Ni—Ti alloy are imparted by at least about 30% cold work and heat treatment at a temperature of at least about 550K.

4. The guide wire device of claim 2, wherein the elevated upper plateau stress and the narrowed stress hysteresis width of the Ni—Ti alloy are imparted by about 40% cold work and heat treatment at a temperature of about 670K to about 725K for about 30 minutes.

5. The guide wire device of claim 1, wherein the elevated upper plateau stress is in a range from about 500 MPa to about 820 MPa.

6. The guide wire device of claim 1, wherein the elevated upper plateau stress is about 550 MPa and the stress hysteresis width is about 150 MPa.

7. The guide wire device of claim 1, wherein the elevated upper plateau stress is about 820 MPa and the stress hysteresis width is about 80 MPa.

8. The guide wire device of claim 1, wherein the Ni—Ti alloy comprises about 54.5 wt % to about 57 wt % Ni and a balance of Ti.

9. The guide wire device of claim 1, wherein the Ni—Ti alloy comprises about 50.2 at % Ni and about 49.8 at % Ti.

10. The guide wire device of claim 1, wherein the distal section includes the Ni—Ti alloy and the proximal section includes at least one of a stainless steel, a superelastic nickel-titanium alloy, or the Ni—Ti alloy.

11. The guide wire device of claim 1, wherein each of the proximal and distal sections are fabricated from the Ni—Ti alloy that exhibits the elevated upper plateau stress of at least about 500 MPa and the narrowed stress hysteresis width of about 250 MPa.

12. The guide wire device of claim 1, further comprising a welded joint joining the proximal and distal sections of the elongate guide wire member to one another.

13. The guide wire device of claim 1, further comprising:
a helical coil section disposed about at least a distal portion of the distal section; and
an atraumatic cap section joined to a distal end of the helical coil section.

14. A method for fabricating a guide wire device, comprising:
fabricating an elongate guide wire member having a proximal section and a distal section, wherein at least one of the proximal section or the distal section includes a nickel-titanium (Ni—Ti) alloy;
cold working at least a portion of the Ni—Ti alloy to yield a cold worked section that exhibits at least about 30% cold work; and
heat treating the cold worked portion to yield a Ni—Ti alloy that exhibits an elevated upper plateau stress of at least about 500 MPa and a narrowed stress hysteresis width of about 250 MPa.

15. The method of claim 14, wherein the heat treating includes heating the cold worked portion at a temperature of at least about 550K for about 1 minute to about 30 minutes.

16. The method of claim 14, wherein the fabricating includes at least one of:
fabricating at least the distal section of the elongate guide wire member from the Ni—Ti alloy;
cold working the distal section of the elongate guide wire member to yield the Ni—Ti alloy distal section that exhibits at least about 30% cold work; or heat treating the distal section at a temperature of at least about 550K for about 10 minutes to about 30 minutes to yield a Ni—Ti alloy distal section having an elevated upper plateau stress of at least about 500 MPa and a narrowed stress hysteresis width of about 250 MPa.

17. The method of claim 14, wherein the fabricating includes:
fabricating the proximal and distal sections of the elongate guide wire member from a Ni—Ti alloy;
cold working at least a portion the elongate guide wire member to yield a cold worked Ni—Ti alloy that exhibits at least about 30% cold work; and
heat treating the elongate guide wire member at a temperature of at least about 550K for about 10 minutes to about 30 minutes to yield a Ni—Ti alloy that exhibits an elevated upper plateau stress of at least about 500 MPa and a narrowed stress hysteresis width of about 250 MPa.

18. The method of claim 14, wherein the cold working and the heat treating yield a Ni—Ti alloy that exhibits an elevated upper plateau stress in a range from about 500 MPa to about 820 MPa.

19. The method of claim 14, wherein the cold working and the heat treating yield a Ni—Ti alloy that exhibits an elevated upper plateau stress in a range of about 500 MPa to about 820 MPa and a narrowed stress hysteresis width in a range of about 250 MPa to about 80 MPa.

20. The method of claim 14, further comprising:
cold working the cold worked section to yield a cold worked Ni—Ti alloy that includes about 30% to about 50% cold work; and
heat treating at least the cold worked section at a temperature of about 550K to about 750K for about 10 minutes to about 30 minutes.

21. The method of claim 14, further comprising:
cold working the cold worked section to yield a cold worked Ni—Ti alloy that includes about 40% cold work; and
heat treating at least the cold worked portion at a temperature of about 670K to about 725K for about 30 minutes.

22. The method of claim 14, wherein the fabricating includes at least one of drawing, swaging, or grinding.

23. The method of claim 14, wherein the cold working includes at least one of drawing, flattening, stamping, rolling, or calendaring.

24. The method of claim 14, wherein the distal section is fabricated from a Ni—Ti alloy and the proximal section is fabricated from at least one of a stainless steel or a Ni—Ti alloy.

25. The method of claim 14, wherein the proximal and distal sections are fabricated from a Ni—Ti alloy.

26. A method for fabricating a guide wire device that includes a Ni—Ti alloy that exhibits an elevated upper plateau stress and a narrowed stress hysteresis width, the method comprising:
providing an elongate guide wire member that includes a proximal section and a distal section, wherein at least the distal section is fabricated from a Ni—Ti alloy;
cold working at least a portion of the distal section to yield a cold worked Ni—Ti alloy that exhibits at least about 30% cold work;
heat treating at least the cold worked portion at a temperature of at least about 550K for about 1 minutes to about 30 minutes to yield a Ni—Ti alloy that exhibits an elevated upper plateau stress of at least about 500 MPa and a narrowed stress hysteresis width of about 250 MPa;
disposing a helical coil section about at least a distal end portion of the distal section;
joining the helical coil to the elongate guide wire member at a proximal location;
forming a rounded cap section on a distal end of the helical coil; and
applying at least one lubricious outer coating layer over at least a portion of the elongate guide wire member to form the guide wire device.

27. The method of claim 25, wherein the proximal and distal sections are fabricated from a Ni—Ti alloy having the elevated upper plateau stress and the narrowed stress hysteresis profile.

28. The method of claim 25, wherein the cold working and the heat treating yield a Ni—Ti alloy that exhibits an elevated upper plateau stress plateau in a range from about 500 MPa to about 820 MPa.

29. The method of claim 25, wherein the cold working and the heat treating yield a Ni—Ti alloy that exhibits an elevated upper plateau stress in a range of about 500 MPa to about 820 MPa and a narrowed stress hysteresis width in a range of about 250 MPa to about 80 MPa.

30. The method of claim 25, further comprising:
cold working the cold worked Ni—Ti alloy portion such that the cold worked Ni—Ti alloy portion exhibits about 30% to about 50% cold work; and
heat treating at least the cold worked portion at a temperature of about 550K to about 750K for about 10 minutes to about 30 minutes.

31. The method of claim 25, further comprising:
cold working the cold worked Ni—Ti alloy portion such that the cold worked Ni—Ti alloy portion exhibits about 40% cold work; and
heat treating at least the cold worked portion at a temperature of about 670K to about 725K for about 30 minutes.
